(19) United States
(12) Patent Application Publication
(10) Pub. No.: US 2016/0069896 A1
(43) Pub. Date: Mar. 10, 2016

(54) DIAGNOSIS OF AUTOIMMUNE DISEASES USING A SPECIFIC ANTIBODY PROFILE

(71) Applicant: YEDA RESEARCH AND DEVELOPMENT CO. LTD., Rehovot (IL)

(72) Inventors: Irun R. COHEN, Rehovot (IL); Eytan DOMANY, Rehovot (IL); Ittai FATTAL, Rehovot (IL); Noam SHENTAL, Rehovot (IL)

(73) Assignee: Yeda Research and Development Co., Ltd., Rehovot (IL)

(21) Appl. No.: 14/652,425

(22) PCT Filed: Dec. 16, 2013
(86) PCT No.: PCT/IL2013/051027

§ 371 Date: Jun. 15, 2015

Related U.S. Application Data
(60) Provisional application No. 61/737,789, filed on Dec. 16, 2012.

(51) Int. Cl. G01N 33/68

(52) U.S. Cl. CPC G01N 33/6854; G01N 2800/104; G01N 2800/60

(57) ABSTRACT

Methods and kits for diagnosing systemic lupus erythematosus (SLE) or scleroderma in a subject are provided. Particularly, the present invention relates to a specific antibody reactivity profile useful in diagnosing SLE or scleroderma in a subject.

### FIELD OF THE INVENTION

The present invention relates to a specific antibody profile useful in diagnosing an autoimmune disorder such as systemic lupus erythematosus (SLE) and scleroderma, in a subject.

### BACKGROUND OF THE INVENTION

Systemic lupus erythematosus (SLE), a prototypic autoimmune disease, is associated with a large spectrum of autoantibodies. IgG antibodies to more than 100 different antigens including DNA, nucleosomes, histones, viral antigens, transcription factors and more have been reported in different SLE patients (Sherer et al., 2004, Semin Arthritis. Rheum. 34:501-37). Surprisingly, there is no serologic diagnosis of SLE and the diagnosis is made on the basis of eleven criteria defined by the American College of Rheumatology (ACR). These criteria include malar rash, discoid rash, photosensitivity, oral ulcers, arthritis, serositis, renal disorder, neurologic disorder, hematologic disorder (e.g., leucopenia, lymphopenia, hemolytic anemia or thrombocytopenia), immunologic disorder and antibody abnormalities (particularly anti-nuclear antibodies (ANA) and anti-DNA antibodies) (Tan et al., 1997, Arthritis Rheum 1997, 40:1725). A subject can be clinically diagnosed with SLE if he meets at least four of the eleven criteria. Nevertheless, SLE is still possible even in case when less then four criteria are present.

While anti-nuclear antibodies and autoantibodies to dsDNA, phospholipids and Sm proteins are among the eleven criteria used for diagnosing SLE (Tan et at, 1997, ibid.), many patients diagnosed with SLE lack these autoantibodies, especially when they are in clinical remission.

International Patent Application Publication No. WO 11/099012, of some the present inventors, relates to methods and kits for diagnosing systemic lupus erythematosus (SLE) in a subject, using a specific antibody profile. The '012 publication discloses patients having, inter alia, increased IgG reactivity to Epstein-Barr Virus (EBV). Additional patents and patent applications disclosing diagnosis of autoimmune diseases using a specific antibody profile include WO 10/055510, WO 12/052994, US 2005/0260770 and U.S. Pat. No. 8,010,298. Further, US Patent Application No. 2012/0122720 relates to recognizing the development of cardiovascular disease, e.g., acute myocardial infarction process in an individual.

Herkel et al. (Journal of Autoimmunity, 2001, 17, 63-69) reported that SLE patients, in addition to anti-DNA, produce antibodies to the carboxy-terminal domain of p53. Notably, the antibody reactivity was limited to the carboxy-terminal domain of p53 that binds damaged DNA, while there was no significant recognition of a control peptide from the amino terminus of p53.

Scleroderma (or systemic sclerosis) is an autoimmune disease that is characterized by endothelial cell damage, fibroblast activation, extracellular matrix (ECM) accumulation and abnormal angiogenesis that carries a high rate of morbidity and mortality. One of the major causes of mortality is fibrosis of lung tissue (interstitial lung disease) and severe pulmonary hypertension. The pathogenesis of scleroderma remains unclear, but is thought to involve an autoimmune response against target organs with early production of autoantibodies and inflammatory mononuclear cell infiltrates followed by loss of organ function and fibrosis. Principal target organs are the skin, the gastrointestinal tract, the lungs and kidneys, although other organs are also frequently involved. Widespread scleroderma can occur with other autoimmune diseases, including SLE.

One of the most difficult challenges in clinical management of complex autoimmune diseases such as SLE or scleroderma is the accurate and early identification of the disease in a patient. There remains a need for improved diagnostic methods and kits useful in diagnosing SLE or scleroderma in a subject.

### SUMMARY OF THE INVENTION

The present invention provides methods and kits for diagnosing an autoimmune disorder, particularly systemic lupus erythematosus (SLE) and/or scleroderma. The present invention further provides antigen probe arrays for practicing such a diagnosis, and antigen probe sets for generating such arrays.

The present invention is based, in part, on the unexpected results obtained when testing the antibody reactivity of SLE patients compared to other autoimmune conditions, particularly scleroderma and pemphigus patients, as well as in comparison to healthy controls. Surprisingly, decreases as well as increases in IgG reactivities to specific Epstein-Barr Virus (EBV) antigen preparations were found in 80% of the SLE patients. Changes in EBV antibodies appeared in SLE patients both positive and negative for anti-dsDNA. Thus, reactivities to EBV antigens can advantageously extend the serologic detection of SLE beyond subjects having anti-dsDNA autoantibodies.

Changes in EBV antibodies were also found in scleroderma patients. However, it was found that SLE patients, and not scleroderma patients, exhibit a unique antibody reactivity profile, including, but not limited to a surprising decrease in IgM reactivities to Glutathione S-Transferase (GST).

Thus, the present invention provides unique antigen-autoantibody reactivity patterns relevant to SLE and scleroderma. In particular embodiments, the present invention provides highly specific, reliable, accurate and discriminatory assays for identifying a subject afflicted with SLE or scleroderma. In exemplary embodiments, the unique antigen-autoantibody reactivity pattern of the present invention characterizes patients who are also negative for anti-dsDNA.

According to a first aspect, the present invention provides a method of diagnosing an autoimmune disease selected from SLE and scleroderma in a subject, the method comprising:
(i) determining the reactivity of IgG antibodies in a sample obtained from the subject to a plurality of antigens selected from the group consisting of: EBVp18, EBVp23, EBNA-1 (EBV Nuclear Antigen 1) and EBVEA (EBV Early Antigen), thereby determining the reactivity pattern of the sample to the plurality of antigens; and
(ii) comparing the reactivity pattern of said sample to a control reactivity pattern;

wherein a significant difference between the reactivity pattern of said sample obtained from the subject compared to the pattern of the control reactivity is an indication that the subject is afflicted with SLE or scleroderma.

According to some embodiments, the method of the present invention is useful in diagnosing SLE in subjects negative for anti-dsDNA (i.e., lacks anti-dsDNA autoantibodies). In one embodiment, said sample obtained from the subject is substantially devoid of antibody reactivity to dsDNA. In another embodiment, said subject is suspected of having an autoimmune disease. In yet another embodiment, said subject is suspected of having SLE or scleroderma.

According to another embodiment, the plurality of antigens comprises a plurality of antigens selected from EBVp18 and at least one antigen selected from EBVp23, EBNA-1 and EBVEA. According to one embodiment, the plurality of antigens comprises EBVp18 and EBVp23. According to additional embodiments, the plurality of antigens comprises at least three antigens. According to specific embodiments, the plurality of antigens comprises EBVp23, EBVp18, and EBNA-1. According to another embodiment, the plurality of antigens comprises EBVp23, EBVp18, EBNA-1 and EBVEA.

According to one embodiment, said reactivity pattern of the sample comprises increased IgG reactivity. According to another embodiment, said reactivity pattern of the sample comprises decreased IgG reactivity. According to yet another embodiment, said reactivity pattern of the sample comprises both increased and decreased IgG reactivities.

According to some embodiments, said increased IgG reactivity is of at least one antigen selected from EBVp23 and EBVEA. According to other embodiments, said decreased IgG reactivity is of at least one antigen selected from EBVp18 and EBNA-1.

According to another embodiment, a reactivity pattern of the sample comprising increased IgG reactivity of at least one antigen selected from EBVp23 and EBVEA, compared to the reactivity pattern of the control sample, is an indication that the subject is afflicted with SLE or scleroderma. According to another embodiment, a reactivity pattern of the sample comprising decreased IgG reactivity is of at least one antigen selected from EBVp18 and EBNA-1 compared to the reactivity pattern of the control sample, is an indication that the subject is afflicted with SLE or scleroderma.

In another embodiment, the method comprises determining the reactivity of IgG and IgM antibodies. In yet another embodiment, said reactivity pattern of the sample comprises both IgG and IgM reactivities.

According to some embodiments, the method further comprises determining the reactivity of antibodies in said sample to at least one antigen selected from Glutathione S-Transferase (GST), FOXp3-p22, buserelin, MOG, HSP60-p26, P53-p10 and p53-p11, or a subset thereof. According to another embodiment, a reactivity pattern of the sample comprising significantly decreased IgM reactivity of GST; increased IgM reactivity of at least one antigen selected from FOXp3-p22, buserelin, MOG; or increased IgG reactivity of at least one antigen selected from FOXp3-p22, MOG, HSP60-p26, P53-p10 and p53-p11, compared to the reactivity pattern of a control sample, is an indication that the subject is afflicted with SLE.

According to another aspect, the present invention provides a method of diagnosing SLE in a subject, the method comprising:
(i) determining the reactivity of IgG and IgM antibodies in a sample obtained from the subject to a plurality of antigens selected from EBVp18, EBVp23, GST, buserelin, FOXp3-p22, MOG, or a subset thereof; thereby determining the reactivity pattern of the sample to the plurality of antigens; and
(ii) comparing the reactivity pattern of said sample to a control reactivity pattern;

wherein a significant difference between the reactivity pattern of said sample obtained from the subject compared to the reactivity pattern of a control sample is an indication that the subject is afflicted with SLE.

According to some embodiments, the plurality of antigens is selected from EBVp18, and at least one antigen selected from EBVp23, GST, FOXp3-p22, buserelin, MOG, HSP60-p26, P53-p10 and p53-p11. According to another embodiment, the plurality of antigens further comprises at least one antigen selected from HSP60-p26, P53-p10, p53-p11, EBNA-1 and EBVEA.

In one embodiment, a reactivity pattern of the sample comprising significantly increased IgG reactivity to at least one antigen selected from EBVp23 FOXp3-p22, MOG, HSP60-p26, EBVEA, P53-p10 and p53-p11, compared to the reactivity pattern of a control sample, is an indication that the subject is afflicted with SLE. In another embodiment, a reactivity pattern of the sample comprising significantly decreased IgG reactivity to EBVp18 or EBNA-1 compared to the reactivity pattern of a control sample is an indication that the subject is afflicted with SLE.

In another embodiment, a reactivity pattern of the sample comprising significantly increased IgM reactivity to at least one antigen selected from FOXp3-p22, buserelin and MOG, compared to the reactivity pattern of a control sample, is an indication that the subject is afflicted with SLE. In another embodiment, a reactivity pattern of the sample comprising significantly decreased IgM reactivity to GST, compared to the reactivity pattern of a control sample, is an indication that the subject is afflicted with SLE.

According to additional embodiments of the methods of the present invention, the sample obtained from the subject is a biological fluid. According to some embodiments, the sample is selected from the group consisting of plasma, serum, blood, cerebrospinal fluid, synovial fluid, sputum, urine, saliva, tears, lymph specimen, or any other biological fluid known in the art. Each possibility represents a separate embodiment of the invention. According to certain embodiments, the sample obtained from the subject is selected from the group consisting of serum, plasma and blood. According to one embodiment, the sample is a serum sample.

According to certain embodiments of the methods of the present invention, the control is selected from the group consisting of a sample from at least one healthy individual, a panel of control samples from a set of healthy individuals, and a stored set of data from healthy individuals. Typically, a healthy individual is a subject not afflicted with SLE (or any other form of lupus). In another embodiment, a healthy individual is a subject not afflicted with an autoimmune disease. In yet another embodiment, a healthy individual is a subject not afflicted with scleroderma.

According to another aspect the present invention provides a kit for the diagnosis of SLE or scleroderma in a subject comprising a plurality of antigens selected from the group consisting of: EBVp23, EBVp18, EBNA-1 and EBVEA, or a subset thereof. In some embodiments, there is provided a kit for the diagnosis of SLE in a subject comprising a plurality of antigens selected from the group consisting of: EBVp23, EBVp18, EBNA-1, EBVEA and GST.

According to another aspect the present invention provides a kit for the diagnosis of SLE in a subject comprising a plurality of antigens selected from EBVp23, EBVp18, GST, FOXp3-p22, buserelin and MOG, or a subset thereof. In some embodiments, the plurality of antigens comprises EBVp23, EBVp18, EBNA-1, EBVEA, GST, FOXp3-p22, buserelin, MOG, HSP60-p26, P53-p10 and p53-p11, or a subset thereof.

According to another aspect, the present invention provides an antigen probe set comprising a plurality of antigen probes selected from the group consisting of: EBVp23, EBVp18, EBNA-1 and EBVEA, or a subset thereof. In some embodiments, there is provided an antigen probe set comprising a plurality of antigen probes selected from the group consisting of: EBVp23, EBVp18, EBNA-1, EBVEA and GST.

According to another aspect, the present invention provides an antigen probe set comprising a plurality of antigen probes selected from EBVp23, EBVp18, GST, FOXp3-p22, buserelin and MOG, or a subset thereof. In one embodiment, the antigen probe set further comprises at least one antigen selected from HSP60-p26, P53-p10 and p53-p11. According to another aspect, the present invention provides an article of manufacture comprising the antigen probe set of the present invention.

According to another aspect, there is provided use of an antigen probe set comprising a plurality of antigen probes selected from the group consisting of: EBVp23, EBVp18, EBNA-1 and EBVEA, for the preparation of a diagnostic kit for diagnosing SLE or scleroderma in a subject. According another embodiment there is provided use of the antigen probe set comprising a plurality of antigen probes selected from the group consisting of: EBVp23, EBVp18, EBNA-1, EBVEA and GST, for the preparation of a diagnostic kit for diagnosing SLE in a subject. According another aspect there is provided use of the antigen probe set comprising a plurality of antigen probes selected from the group consisting of: EBVp23, EBVp18, GST, FOXp3-p22, buserelin and MOG, or a subset thereof, for the preparation of a diagnostic kit for diagnosing SLE in a subject.

Said diagnostic kit is, in some embodiments, useful for determining the reactivity of antibodies in a sample, thereby determining the reactivity pattern of the sample to said plurality of antigens. In some embodiments, a significant difference between the reactivity pattern of said sample compared to a reactivity pattern of a control sample is an indication for SLE.

Other objects, features and advantages of the present invention will become clear from the following description and drawings.

### BRIEF DESCRIPTION OF THE DRAWINGS

FIG. 1—IgG and IgM reactivities to selected antigens (IgG reactivates to DNAds, EBV, MOG, p53P11 and BMP4; IgM reactivities to C10G10methyl, DNAds and GST) in healthy controls and in SLE and scleroderma (SSc) patients. The relative amount of antibody reactivity is shown on the Y axis. The X axis orders the subjects according to their relative reactivity. The horizontal lines mark the value that differed SLE patients from controls in a PPV≧90%. Each spot represents a single subject.

FIG. 2—IgG reactivities to EBV antigens (EBV, EBVp23, EBVp18 and EBNA-1) in healthy controls and in SLE and scleroderma (SSc) patients. Note that subgroups of SLE patients show increased reactivities to EBV and EBVp23 or decreases to EBVp18 or EBNA1. The relative amount of antibody reactivity is shown on the Y axis. The X axis orders the subjects according to their relative reactivity. The horizontal lines mark the value that differed SLE patients from controls in a PPV≧90%. Each spot represents a single subject.

FIG. 3—SLE detection rate of the IgG significant antigens (MOG, FOXp3-p22, HA, BMP4, HSP60-p26, p53-p10, p53p11 and IGFBP1) compared to dsDNA, and EBV antigens, as well as their combinations. The SLE patients detected by the IgG significant antigens mostly overlapped with those detected by dsDNA and added little to the detection rate of anti-dsDNA (dark gray rectangle). In contrast, SLE patients detected by EBV antigens only partly overlapped with those detected by anti-dsDNA and significantly added to the detection rate of dsDNA (bright gray rectangle).

### DETAILED DESCRIPTION OF THE INVENTION

The present invention provides methods of diagnosing an autoimmune disease or disorder, specifically systemic lupus erythematosus (SLE) and/or scleroderma, in a subject. The present invention further provides antigen probe arrays for practicing such a diagnosis, and identifies specific antigen probe sets for generating such arrays.

Various antigens previously disclosed as capable of characterizing SLE patients, were found as not significantly adding to the detection rate of dsDNA. Unexpectedly however, IgG reactivities to EBV antigens (e.g., EBVp18 and EBVp23) were found not to overlap with IgG reactivities to dsDNA. As exemplified herein below, a large prevalence of pathological serology to EBV antigens in SLE patients, at least one of the following was found in more than 85% of the SLE patients examined: increased IgG reactivities to EBVEA (EBV Early Antigen) or EBVp23, or decreased IgG reactivities to EBVp18 or EBVEBNA (EBV Nuclear Antigen or interchangeably EBNA-1). Interestingly, some of the SLE patients were negative for dsDNA but positive for at least one of the EBV antigens.

The IgG reactivities to the EBV antigens were not confined to SLE patients, but appeared in scleroderma patients too, suggesting a general role of EBV in autoimmune diseases, and particularly indicate a role in SLE and scleroderma. The present invention further discloses that SLE patients may be serologically differentiated from scleroderma patients. It is disclosed for the first time that decreased IgM reactivities to Glutathione S-Transferase (GST) and (CpG) repeats, among other antigens, constitute a unique serological signature for SLE patients. SLE patients, and not scleroderma patients, exhibited a decrease in IgM reactivities to Glutathione S-Transferase (GST), an increase in IgM reactivities to glucose-6-phosphate isomerase (132GP1) and increases in IgG reactivities to FOXp3-p22, HSP60-p26, P53-p10, p53-p11, β2GP1, HGF, MOG, BMP4, HA, dsDNA, ssDNA and Sm. Thus, in some embodiments, the present invention provides assays for discriminating and differentiating between subjects afflicted with SLE and/or scleroderma, using at least one or a plurality of antigen selected from GST, FOXp3-p22, HSP60-p26, P53-p10, p53-p11, β2GP1, HGF, MOG, BMP4, HA, dsDNA, ssDNA and Sm, or a subset or combination thereof.

The present invention provides, in some embodiments, unique antigen-autoantibody reactivity patterns particularly relevant to SLE and scleroderma. As exemplified herein below, SLE patients have at least one of 3 serological signatures: 1. Increased IgG reactivities to a large spectrum of proteins, peptides, and hyaluronic acid from both human and bacteria that mostly overlapped with the dsDNA reactivities; 2. Increases and decreases in IgG reactivities to EBV antigens; 3. Decreases in IgM reactivities to GST and/or (CpG) repeats. These serological signatures partially overlap and at least one of them was found in 96% of the SLE patients.

In some embodiment, there is provided a plurality of antigens for discriminating SLE and healthy controls. In additional embodiments, there is provided a plurality of antigens for discriminating SLE and scleroderma patients.

Without wishing to be bound by any particular theory or mechanism of action, the invention is based in part on the finding that the antibody reactivity profile in serum of SLE patients was clearly distinct from healthy control individuals. Although serum autoantibodies have been extensively investigated in SLE, the unique antibody immune signatures as described herein have not been described before. Advantageously, the unique antibody signatures of the present invention provide highly sensitive and specific assays for diagnosing SLE. Further, the antibody signatures of the present invention characterize patients who are also negative for anti-dsDNA.

In additional embodiments, the method of the invention comprises determining the reactivity of IgM antibodies to at least one antigen selected from GST and (CpG) repeats, in a sample obtained from a subject (suspected of having SLE or scleroderma), wherein a significant decrease in the IgM reactivity to at least one antigen compared to a control sample is an indication that the subject is afflicted with SLE. In another embodiment, the method of the invention comprises determining the reactivity of IgM antibodies to GST and (CpG) repeats.

In a further embodiment, the method of the invention comprises determining the reactivity of IgM antibodies to GST. In specific embodiments, a significant decrease in the IgM reactivity of GST compared to the reactivity pattern of a control sample is an indication that the subject is afflicted with SLE. In yet another embodiment, the method of the invention comprises determining the reactivity of IgM antibodies to (CpG) repeats. The term “CpG” as used herein, refers to repeats of cytosine and guanine linked by a phosphodiester bond. In some embodiments, a CpG repeat refers to repeats of about 10 cytosines and 10 guanines. In another embodiment, a CpG repeat antigen comprises or consists of the oligonucleotide sequence as set forth in SEQ ID NO: 7.

As exemplified herein below, antigen analysis of autoantibodies (e.g., using microarray analysis) can identify serum autoantibody patterns associated with SLE or scleroderma; the signatures were based on collective autoantibody patterns, not single autoantibody reactivities. These informative patterns included decreases and increases of IgG autoantibodies as well as decreases IgM autoantibodies, relative to those found in healthy controls.

In a particular embodiment, the method comprises:

(i) obtaining a sample from a subject;

(ii) determining the reactivity of IgG antibodies in the sample to a plurality of antigens selected from the group consisting of: EBVEA (EBV Early Antigen), EBVp23, EBVp18 and EBNA-1 (EBV Nuclear Antigen); and optionally determining the reactivity of IgM antibodies in the sample to a GST antigen; thereby determining the reactivity pattern of the sample to the plurality of antigens; and

(iii) comparing the reactivity pattern of said sample to a control reactivity pattern;

wherein a significant difference between the reactivity pattern of said sample obtained from the subject compared to the reactivity pattern of a control sample is an indication that the subject is afflicted with SLE or scleroderma.

In particular embodiments, a significant increase between the reactivity pattern of the IgG antibodies to at least one antigen selected from EBVEA, EBVp23, in said sample obtained from the subject compared to the control reactivity pattern is an indication that the subject is afflicted with SLE or scleroderma, and/or a significant decrease between the reactivity pattern of the IgG antibodies to at least one antigen selected from EBVp18 and EBNA-1, in said sample obtained from the subject compared to the control reactivity pattern is an indication that the subject is afflicted with SLE or scleroderma. In another particular embodiment, a significant decrease between the reactivity pattern of the IgM antibodies to GST, in said sample obtained from the subject compared to the control reactivity pattern is an indication that the subject is afflicted with SLE.

As used herein, the “reactivity of antibodies in a sample” to “a plurality of antigens” refers to the immune reactivity of each antibody in the sample to a specific antigen selected from the plurality of antigens. The immune reactivity of the antibody to the antigen, i.e. its ability to specifically bind the antigen, may be used to determine the amount of the antibody in the sample. The calculated levels of each one of the tested antibodies in the sample are selectively referred to as the reactivity pattern of the sample to these antigens.

The reactivity pattern of the sample reflects the levels of each one of the tested antibodies in the sample, thereby providing a quantitative assay. In a preferred embodiment, the antibodies are quantitatively determined.

A “significant difference” between reactivity patterns refers, in different embodiments, to a statistically significant difference, or in other embodiments to a significant difference as recognized by a skilled artisan. In yet another preferred embodiment, a significant (quantitative) difference between the reactivity pattern of said sample obtained from the subject compared to the control reactivity pattern is an indication that the subject is afflicted with SLE and in some embodiments scleroderma. In specific embodiments, up-regulation of the reactivity of an antibody in a sample to an antigen refers to an increase (i.e., elevation) of about at least two, about at least three, about at least four, or about at least five times higher (i.e., greater) than the reactivity levels of the antibody to the antigen in the control. In another embodiment, down-regulation of the reactivity of an antibody in a sample to an antigen refers to a decrease (i.e., reduction) of about at least two, about at least three, about at least four, or about at least five times lower than the reactivity levels of the antibody to the antigen in the control.

In particular embodiments, said significant difference is determined using a cutoff of a positive predictive value (PPV) of at least 85%, preferably at least 90%. Determining a PPV for a selected marker (e.g., an antigen) is well known to the ordinarily skilled artisan and is exemplified in the methods described below. Typically, positivity for an antigen is determined if it detected above 10% of the subjects in a specific study subgroup using a selected cutoff value, such as PPV≧90%. For example, antigen i is determined to specifically characterize group A if it detected at least 10% of the subjects in group A with a PPV≧90% when compared to a different test group B. Subjects in group A that are above the cutoff of PPV≧90% for antigen i are considered to be positive for antigen i.

An antibody “directed to” an antigen, as used herein is an antibody which is capable of specifically binding the antigen. Determining the levels of antibodies directed to a plurality of antigens includes measuring the level of each antibody in the sample, wherein each antibody is directed to a specific antigen, including but not limited to, an antigen selected from: EBNA-1, EBVp23, EBVp18, EBVEA and GST. This step is typically performed using an immunoassay, as detailed herein.

In other embodiments, determining the reactivity of antibodies in said sample to said plurality of antigens, (and the levels of each one of the tested antibodies in the sample) is performed by a process comprising:
(i) contacting the sample, under conditions such that a specific antigen-antibody complex may be formed, with an antigen probe set comprising said plurality of antigens, and
(ii) quantifying the amount of antigen-antibody complex formed for each antigen probe.

The amount of antigen-antibody complex is indicative of the level of the tested antibody in the sample (or the reactivity of the sample with the antigen).

In another embodiment the method comprises determining the reactivity of at least one IgG antibody and at least one IgM antibody in said sample to said plurality of antigens. In another embodiment, the method comprises determining the reactivity of a plurality of IgG antibodies and at least one IgM antibodies in said sample to said plurality of antigens.

Typically, determining the reactivity of antibodies in the sample to the plurality of antigens is performed using an immunoassay. Advantageously, the plurality of antigens may be used in the form of an antigen array.

Antigen Probes and Antigen Probe Sets

According to further embodiments, the invention provides antigen probes and antigen probe sets useful for diagnosing SLE or scleroderma, as detailed herein.

According to the principles of the invention, the invention further provides a plurality of antigens also referred to herein as antigen probe sets. These antigen probe sets comprising a plurality of antigens are reactive specifically with the sera of subjects having SLE or scleroderma. According to the principles of the invention, the plurality of antigens may advantageously be used in the form of an antigen array. According to some embodiments the antigen array is conveniently arranged in the form of an antigen chip.

A “probe” as used herein means any compound capable of specific binding to a component. According to one aspect, the present invention provides an antigen probe set comprising a plurality of antigens selected from the group consisting of: EBVp23, EBVp18, EBNA-1, EBVEA and GST or any combinations thereof. According to certain embodiments, the antigen probe set comprises a subset of the antigens of the present invention. In a particular embodiment, the subset of antigen comprises or consists of: EBVp23, EBVp18, EBNA-1 and GST. In another particular embodiment, the subset of antigen comprises or consists of: EBVp23, EBVp18 and EBNA-1. In yet another particular embodiment, the subset of antigen comprises or consists of: EBVp23, EBVp18 and GST.

According to additional embodiments, the plurality of antigens comprises EBVp23 and at least one antigen selected from EBVp18, EBNA-1, EBVEA and GST. According to another embodiment, the plurality of antigens comprises EBVp23 and at least one antigen selected from EBVp18, EBNA-1, EBVEA, HSP60-p26, P53-p10, p53-p11, FOXp3-p22, buserelin and MOG. According to yet another embodiment, the plurality of antigens comprises EBVp23 and at least one antigen selected from EBVp18, and EBNA-1. According to another embodiment, the plurality of antigens comprises EBVp23 and EBVp18.

According to additional embodiments, the plurality of antigens comprises EBVp18 and at least one antigen selected from EBVp23, EBNA-1, EBVEA and GST. According to another embodiment, the plurality of antigens comprises EBVp18 and at least one antigen selected from EBVp23, EBNA-1, EBVEA, HSP60-p26, P53-p10, p53-p11, FOXp3-p22, buserelin and MOG. According to yet another embodiment, the plurality of antigens comprises EBVp18 and at least one antigen selected from EBVp23, and EBNA-1.

The reactivity of antibodies to the plurality of antigens of the invention may be determined according to techniques known in the art. Further, the antigens used in the present invention are known in the art and are commercially available, e.g., from Prospec or Sigma-Aldrich.

EBV Antigens

EBV (Epstein Barr virus) is a herpes virus also termed human herpes virus 4 (HHV-4) that can cause a large spectrum of clinical manifestations, from infectious mononucleosis to Burkitt's lymphoma. The hallmark of the pathogenesis of EBV is the establishment of latency in B cells. In the latent phase EBV genome can encode proteins such as latent membrane protein 1, an EBV oncoprotein that can induce the B-cell activating factor BAFF, that can activate self-reactive B cells and induce a lupus-like disease in transgenic mice (Niller et al. Autoimmunity. 2008 May; 41(4):298-328). These EBV infected B cells can escape the immune system surveillance and maintain chronic pathological function, indeed it was found that SLE patients have increased viral loads and a defective control of latent EBV infection (Kang I, et al. J Immunol. 2004 Jan. 15; 172(2):1287-94). Without wishing to be bound to any theory or mechanism of action, the increased IgG reactivities to EBV in SLE patients can represent a state of chronic infection and on the other hand the decreased IgG reactivities may be linked to a defective immune reaction to the virus.

The reactivity of antibodies to the plurality of the EBV antigens may be determined according to techniques known in the art. In some embodiments, at least one EBV antigen is fused to a GST tag, preferably at the N-terminus.

EBVp18

The EBVp18 antigen is known in the art to contain the HHV-4 p18 region, having the amino acid sequence as set forth in SEQ ID NO: 1 (ASAGTGALASSAPSTAVAQSATPSVSSSISSLRAATSGATAAASAAAAVDTGSGGG GQPHDTAPRGARKKQ). In some embodiments, the EBVp18 antigen comprises amino acids 1-119 of the EBV Capsid Antigen. In another embodiment, the EBVp18 antigen comprises the amino acid sequence as set forth in SEQ ID NO: 1. In yet another embodiment, the EBVp18 antigen consists of the amino acid sequence as set forth in SEQ ID NO: 1.

EBVp23

EBVp23 is a viral late complex associated with virion particles and consists of two gene products, BFRF3 (p18) and BLRF2 (p23). The EBVp23 antigen is known in the art as a recombinant EBV protein comprising the EBV p23 fragment, amino acids 1-162 of the EBV Capsid Antigen. In some embodiments, the EBVp23 antigen comprises the amino acid sequence as set forth in SEQ ID NO: 2 (SAPRKVRLPSVKAVDMSMEDMAARL ARLESENKALKQQVLRGGACASSTSVPSAPVPPPEPLTARQREVMITQATGRLASQ AMKKIEDKVRKSVDGVTTRNEMENILQNLTLRIQVSMLGAKGQPSPGEGTRPRESN DPNATRRARSRSRGREAKKVQISD). In yet another embodiment, the EBVp23 antigen consists of the amino acid sequence as set forth in SEQ ID NO: 2.

EBNA-1

EBV EBNA-1 (also termed herein EBVEBNA) plays an essential role in replication and partitioning of viral genomic DNA during latent viral infection. During this phase, the circular double-stranded viral DNA undergoes replication once per cell cycle and is efficiently partitioned to the daughter cells. In a particular embodiment, the EBV EBNA-1 contains the HHV-4 EBNA regions, amino acids 1-90 (set forth in SEQ ID NO: 3; MSDEGPGTGPGNGLGEKGDTSGPEGSGGSGPQRRGGDNHGRGRGRGRGRGGGRP GAPGGSGSGPRHRDGVRRPQKRPSCIGCKGTHGGTG) and 408-498 (set forth in SEQ ID NO: 4 PVGEADYFEYHQEGGPDGEPDVPPGAIEQGPADDPGEGPSTGP RGQGDGGRRKKGGWFGKHRGQGGSNPKFENIAEGLRALLARSHVERTTD). In yet another embodiment, the EBV EBNA-1 comprises the amino acid sequence as set forth in SEQ ID NO: 5 (MSDEGPGTGPGNGLGEKGDTSGPEGSGGSGPQRRGGDNHGRGR GRGRGRGGGRPGAPGGSGSGPRHRDGVRRPQKRPSCIGCKGTHGGTGPVGEADYF EYHQEGGPDGEPDVPPGAIEQGPADDPGEGPSTGPRGQGDGGRRKKGGWFGKHR GQGGSNPKFENIAEGLRALLARSHVERTTD). In yet another embodiment, the EBNA-1 antigen consists of the amino acid sequence as set forth in SEQ ID NO: 5.

EBV Early Antigen

The EBV Early Antigen (EBVEA) is known in the art to contain the HHV-4 Early Antigen Type D, C-terminus regions amino acids 306-390. In some embodiments, the EBVEA comprises the amino acid sequence as set forth in SEQ ID NO: 6 (ASEP EDKSPRVQPLGTGLQQRPRHTVSPSPSPPPPPRTPTWESPARPETPSPAIPSHSSNTAL ERPLAVQLARKRTSSEARQKQ). In yet another embodiment, the EBVEA antigen consists of the amino acid sequence as set forth in SEQ ID NO: 6.

GST

Glutathione S-transferases (GST) are a family of proteins that catalyze the conjugation of reduced glutathione with a variety of hydrophobic chemicals containing electrophilic centers. The GST antigen used in the examples section herein below was purchased from Sigma-Aldrich (catalog No. G8642), and has the CAS Number of 50812-37-8. In one embodiment, the GST antigen of the invention has the UniProtKB ID of P09488. In some embodiments, the GST antigen comprises or consists of the amino acid sequence as set forth in SEQ ID NO: 8. In another embodiment, the GST antigen of the invention has the UniProtKB ID of P09211. In some embodiments, the GST antigen comprises or consists of the amino acid sequence as set forth in SEQ ID NO: 9.

Buserelin

Buserelin belongs to the group of gonadotrophin releasing hormone (gonadorelin) analogues (LHRH agonist). It acts on the pituitary gland which controls the amount of many different types of hormones (chemical messengers). It alters the amount of hormones, particularly the estrogens and androgens. This alteration of hormone levels can be exploited to treat cancers of the prostate gland, which are stimulated to grow by testosterone. Buserelin lowers the levels of testosterone, which starves the tumor of testosterone and causes it to shrink. Buserelin contains 9 amino acids Glu-His-Trp-Ser-Tyr-D-Ser(tBu)-Leu-Arg-Pro-NHEt and has a molecular weight of 1239.44 Dalton. The Buserelin antigen used in the examples section herein below was purchased from Prospec (catalog No. HOR-255). In some embodiments, the buserelin antigen comprises or consists of the amino acid sequence as set forth in SEQ ID NO: 10.

Myelin Oligodendrocyte Glycoprotein (MOG)

MOG is a transmembrane protein expressed on the surface of oligodendrocyte cell and on the outermost surface of myelin sheaths. MOG comprises about 0.1% of total CNS myelin protein. The MOG gene is a member of the immunoglobulin gene superfamily and is found within the MHC. The MOG gene is found on chromosome 6p21.3-p22. Myelin Oligodendrocyte Glycoprotein is a glycoprotein thought to be significant in the process of myelinization of nerves in the central nervous system (CNS). MOG peptide (35-55) is highly encephalitogenic and can induce strong T and B cell responses. A single injection of this peptide produces a relapsing-remitting neurologic disease with extensive plaque-like demyelination. Because of the clinical, histophathologic, and immunologic similarities with multiple sclerosis (MS), the MOG induced demyelinating encephalomyelitis may serve as a model for investigating MS. The MOG antigen used in the examples section herein below was purchased from Prospec (catalog No. PRO-371). In some embodiments, the MOG antigen comprises or consists of the amino acid sequence as set forth in SEQ ID NO: 11.

Bone Morphogenetic Protein-4

The protein encoded by this gene is a member of the bone morphogenetic protein family which is part of the transforming growth factor-beta superfamily. The superfamily includes large families of growth and differentiation factors. Bone morphogenetic proteins were originally identified by an ability of demineralized bone extract to induce endochondral osteogenesis in vivo in an extraskeletal site. This particular family member plays an important role in the onset of endochondral bone formation in humans, and a reduction in expression has been associated with a variety of bone diseases, including the heritable disorder Fibrodysplasia Ossificans Progressiva. Alternative splicing in the 5′ untranslated region of this gene has been described and three variants are described, all encoding an identical protein. The BMP4 antigen used in the examples section herein below was purchased from Prospec (catalog No. CYT-361). The BMP-4 antigen is in some embodiments, human recombinant such as produced in E. Coli is a monomeric, non-glycosylated, polypeptide chain containing 116 amino acids and having a molecular mass of 13009 Dalton. In one embodiment, the BMP4 antigen comprises or consists of the amino acid sequence as set forth in SEQ ID NO: 12 (SPKHHSQRAR KKNKNCRRHS LYVDFSDVGW NDWIVAPPGY QAFYCHGDCP FPLADHLNST NHAIVQTLVN SVNSSIPKAC CVPTELSAIS MLYLDEYDKV VLKNYQEMVV EGCGCR).

FOXp3-p22

FOX (Forkhead box) Protein 3 (also known as scurfin) is a protein involved in immune system responses. Human FOXp3 variant is 454 amino acids long (UniProtKB: B7ZLG1). The FOXp3-p22 antigen of the invention is a fragment of the FOXp3, particularly of amino acids 290-304. In one embodiment, FOXp3-p22 comprises the amino acid sequence as set forth in SEQ ID NO: 13 (TKASSVASSQGPVVP), or an analog or fragment thereof. In another embodiment, FOXp3-p22 consists of the amino acid sequence as set forth in SEQ ID NO: 13. In another embodiment, FOXp3-p22 comprises or consists of the amino acid sequence as set forth in SEQ ID NO: 14 (TKASSVASSDKGSCC).

HSP60-p26

Heat shock protein (HSP)60-p26 is a peptide derived from HSP60, particularly amino acids 376-395 of HSP60 (UniProtKB:P63038). In one embodiment, HSP60-p26 comprises the amino acid sequence as set forth in SEQ ID NO: 15 (EQLDITTSEYEKEKLNERLA), or an analog or fragment thereof. In another embodiment, HSP60-p26 consists of the amino acid sequence as set forth in SEQ ID NO: 15.

P53-p10 and p53-p11

p53-p10 and p53-p11 peptides are derived from p53, particularly from a fragment of p53 having the amino acid sequence identified by UniProtKB: A5JTV6 (YSPPLNKLFC QLAKTCPVQL WVSATPPAGS RVRAMAIYKK SQHMTEVVRR CPHHERCSD) as set forth in SEQ ID NO: 16.

In one embodiment, p53-p10 comprises the amino acid sequence as set forth in SEQ ID NO: 17 (KTCPVQLWVSATPPAGSRVR), or an analog or fragment thereof. In another embodiment, p53-p10 consists of the amino acid sequence as set forth in SEQ ID NO: 17.

In another embodiment, p53-p11 comprises the amino acid sequence as set forth in SEQ ID NO: 18 (GSRVRAMAIYKKSQHMTEVV), or an analog or fragment thereof. In another embodiment, p53-p11 consists of the amino acid sequence as set forth in SEQ ID NO: 18.

Preferably, the plurality of antigens of the methods and kits of the invention comprises a set of the antigens as disclosed herein. Yet in other embodiments, the plurality of antigens (or the antigen probe set) comprises or consists of a subset thereof, e.g. at least 3, 4, 5, 6, 7, 8, 9 or 10 different antigens, each selected from the antigens of the present invention. Each possibility represents a separate embodiment of the invention. Such subsets may be selected so as to result in optimal sensitivity and/or specificity of the diagnostic assay. In other embodiments, the probe set comprises up to 6, 7, 8, 9, 10, or in other embodiments up to 15, 20, 30, 40 or 50 different antigens.

In some embodiments antigen probe set of the invention, the plurality of antigens consists of: EBVp23, EBVp18, EBNA-1, EBVEA and GST. In additional embodiments, the plurality of antigens consists of: EBVp23, EBVp18, EBNA-1 and GST. In yet an additional embodiment, the plurality of antigens consists of: EBVp23, EBVp18, EBNA-1 and EBVEA. In another embodiment, the plurality of antigens consists of: EBVp23, EBVp18 and EBNA-1.

As exemplified herein below, a subject suspected of having SLE can be differentiated from healthy controls and from scleroderma patients by assaying and determining IgG and/or IgM antibody reactivities in a sample obtained from said subject (Table 1). According to additional embodiments the antigen probe set of the invention further comprise at least one antigen selected from the group consisting of: hsp60-p17a, hsp60-p26, p53p11, p53p10, buserelin, FOXp3-p22, Sm, MOG (myelin oligo-dendrocyte), β2GP1, dsDNA, ssDNA, HA (human), HA (streptococcus), BMP4 (Bone morphogenic protein 4), IGFBP1 (Insulin growth factor binding protein 1), HGF, hsp60p18, IgM, La and, or a subset or combination thereof. Each possibility represents a separate embodiment of the invention.

In some embodiments with respect to diagnosing SLE, said method comprises:
(i) determining the reactivity of IgG and IgM antibodies in a sample obtained from the subject to a plurality of antigens selected from the group consisting of: EBVp23, EBVp18, EBNA-1, EBVEA, GST, hsp60-p26, p53p11, p53p10, buserelin, FOXp3-p22, Sm, MOG, dsDNA, ssDNA, HA (human), HA (streptococcus), BMP4, IGFBP1, HGF, β2GP1, hsp60p18, or a subset thereof; thereby determining the reactivity pattern of the sample to the plurality of antigens; and
(ii) comparing the reactivity pattern of said sample to a control reactivity pattern;

wherein a significant difference between the reactivity pattern of said sample obtained from the subject compared to the reactivity pattern of a control sample is an indication that the subject is afflicted with SLE.

In some embodiments, said method comprises determining the IgG reactivity of antibodies in the sample to a plurality of antigens selected from EBVp23, EBNA-1, EBVp18, EBVEA, hsp60-p26, p53p10, p53p11, MOG, FOXp3-p22, HA (human), HA (streptococcus), ssDNA, dsDNA, BMP4, IGFBP1, or a subset thereof; thereby determining the reactivity pattern of the sample to the plurality of antigens; wherein a significant difference between the reactivity pattern of said sample obtained from the subject compared to the reactivity pattern of a control sample is an indication that the subject is afflicted with SLE. In particular embodiments, a significant increase in IgG reactivates of a plurality of antigens selected from EBVp23, EBVEA, hsp60-p26, p53p10, p53p11, MOG, FOXp3-p22, HA (human), HA (streptococcus), ssDNA, dsDNA, BMP4, IGFBP1, or a subset thereof, compared to control is an indication that the subject is afflicted with SLE. In yet another particular embodiment, a significant decrease in IgG reactivates of EBNA-1 and/or EBVp18 compared to control is an indication that the subject is afflicted with SLE.

In another embodiment, said method comprises determining the IgM reactivity of antibodies in the sample to a plurality of antigens selected from buserelin, FOXp3-p22, hsp60p18, MOG, BMP4, β2GP1, dsDNA, ssDNA, HA (human), Sm and GST, or a subset thereof; thereby determining the reactivity pattern of the sample to the plurality of antigens; wherein a significant difference between the reactivity pattern of said sample obtained from the subject compared to the reactivity pattern of a control sample is an indication that the subject is afflicted with SLE. In particular embodiments, a significant increase in IgM reactivates of a plurality of antigens selected buserelin, FOXp3-p22, hsp60p18, MOG, BMP4, β2GP1, dsDNA, ssDNA, HA (human) and Sm, or a subset thereof, compared to control is an indication that the subject is afflicted with SLE. In yet another particular embodiment, a significant decrease in IgM reactivates of GST compared to control is an indication that the subject is afflicted with SLE.

According to some embodiments of the methods and antigen probe set of the invention, the plurality of antigens comprise at least one antigen, or a plurality of antigens selected from the group consisting of buserelin, FOXp3-p22, HA (human), Sm, MOG, BMP4, HA (streptococcus), hsp60-p26, p53p11, p53p10 and IGFBP, or a subset thereof.

According to some embodiments, the plurality of antigens further comprise at least one antigen selected from buserelin, FOXp3-p22, HA (human), Sm, MOG, BMP4 or a subset thereof. As exemplified herein below, SLE subjects showed increased IgM reactivates towards said antigens (Table 1).

According to additional embodiments, the plurality of antigens comprise at least one antigen selected from HA (streptococcus), HA (human), FOXp3-p22, MOG, BMP4, hsp60-p26, p53p11, p53p10 and IGFBP, or a subset thereof. As exemplified herein below, SLE subjects showed increased IgG reactivates towards said antigens (Table 1).

In yet another embodiment, the methods and antigen probe set of the invention comprise a plurality of antigens selected from Table 1. In a particular embodiment, IgG and/or IgM reactivity with each antigen as indicated in Table 1 differentiates SLE subjects from healthy subjects or scleroderma subject.

According to an additional embodiment, the present invention provides a method of diagnosing SLE in a subject, the method comprising:
(i) determining the reactivity of IgG and IgM antibodies in a sample obtained from the subject to a plurality of antigens selected from EBVp18, EBVp23, EBNA-1, EBVEA and GST; thereby determining the reactivity pattern of the sample to the plurality of antigens; and
(ii) comparing the reactivity pattern of said sample to a control reactivity pattern;

wherein a significant difference between the reactivity pattern of said sample obtained from the subject compared to the reactivity pattern of a control sample is an indication that the subject is afflicted with SLE.

According to some embodiments, the method comprises determining the reactivity of IgG antibodies in the sample obtained from the subject to a plurality of antigens selected from the group consisting of: EBVp23, EBVp18, EBNA-1 and EBVEA. In a specific embodiment, a significant increase in the IgG reactivity to EBVp23 and/or EBVEA is an indication that the subject is afflicted with SLE. In another specific embodiment, a significant decrease in the IgG reactivity to EBVp18 and/or EBNA-1 is an indication that the subject is afflicted with SLE. According to another embodiment, the method comprises determining the reactivity of IgM antibodies in the sample obtained from the subject to GST. In a specific embodiment, a significant decrease in the IgM reactivity to GST is an indication that the subject is afflicted with SLE.

Furthermore, as exemplified herein below, SLE patients can be differentiated from scleroderma patients, and vice versa, by assaying and determining IgG and/or IgM antibody reactivities to dsDNA, GST, Topoisomerase and/or Centromere B. In some embodiment, the invention provides methods and antigen probe set for differentiating SLE patients from scleroderma patients using a plurality of antigen selected from dsDNA, GST, Topoisomerase and Centromere B, or a subset thereof.

In some embodiments, a reactivity pattern of the sample comprising significantly increased IgG reactivity to at least one antigen selected from Topoisomerase or Centromere B, compared to the reactivity pattern of a control sample (e.g., from an SLE patient), is an indication that the subject is afflicted with scleroderma. In additional embodiments, a reactivity pattern of the sample comprising significantly decreased IgG reactivity to at least one antigen selected from Topoisomerase or Centromere B, compared to the reactivity pattern of a control sample (e.g., from an SSc patient), is an indication that the subject is afflicted with SLE.

In additional embodiments, a reactivity pattern of the sample comprising significantly decreased IgG reactivity to dsDNA or significantly increased IgM reactivity to GST, compared to the reactivity pattern of a control sample (e.g., from an SLE patient), is an indication that the subject is afflicted with scleroderma. In another embodiment, a reactivity pattern of the sample comprising significantly increased IgG reactivity to dsDNA or significantly decreased IgM reactivity to GST, compared to the reactivity pattern of a control sample (e.g., from an SSc patient), is an indication that the subject is afflicted with SLE.

Antigen probes to be used in the assays of the invention may be purified or synthesized using methods well known in the art. For example, an antigenic protein or peptide may be produced using known recombinant or synthetic methods, including, but not limited to, solid phase (e.g. Boc or f-Moc chemistry) and solution phase synthesis methods (Stewart and Young, 1963; Meienhofer, 1973; Schroder and Lupke, 1965; Sambrook et al., 2001). One of skill in the art will possess the required expertise to obtain or synthesize the antigen probes of the invention. The antigen probes are also commercially available, e.g. from Prospec (Ness-Ziona, Israel).

It should be noted, that the invention utilizes antigen probes as well as homologs, fragments and derivatives thereof, as long as these homologs, fragments and derivatives are immunologically cross-reactive with these antigen probes. The term “immunologically cross-reactive” as used herein refers to two or more antigens that are specifically bound by the same antibody. The term “homolog” as used herein refers to a peptide which having at least 70%, at least 75%, at least 80%, at least 85% or at least 90% identity to the antigen's amino acid sequence. Cross-reactivity can be determined by any of a number of immunoassay techniques, such as a competition assay (measuring the ability of a test antigen to competitively inhibit the binding of an antibody to its known antigen).

The term “fragment” as used herein refers to a portion of a polypeptide, or polypeptide analog which remains immunologically cross-reactive with the antigen probes, e.g., to recognize immunospecifically the target antigen. The fragment may have the length of about 40%, about 50%, about 60%, about 70%, about 80%, about 85%, about 90% or about 95% of the respective antigen.

The term peptide typically refers to a polypeptide of up to about 50 amino acid residues in length. According to particular embodiments, the antigenic peptides of the invention may be about 10-100, 10-80, 10-75, 10-50 or about 10-30 amino acids in length.

The term encompasses native peptides (including degradation products, synthetically synthesized peptides, or recombinant peptides), peptidomimetics (typically, synthetically synthesized peptides), and the peptide analogues peptoids and semipeptoids, and may have, for example, modifications rendering the peptides more stable while in a body or more capable of penetrating into cells. Such modifications include, but are not limited to: N-terminus modifications; C-terminus modifications; peptide bond modifications, including but not limited to CH₂—NH, CH₂—S, CH₂—S═O, O═C—NH, CH₂—O, CH₂—CH₂, S═C—NH, CH═CH, and CF═CH; backbone modifications; and residue modifications.

The antigens of the invention may be used having a terminal carboxy acid, as a carboxy amide, as a reduced terminal alcohol or as any pharmaceutically acceptable salt, e.g., as metal salt, including sodium, potassium, lithium or calcium salt, or as a salt with an organic base, or as a salt with a mineral acid, including sulfuric acid, hydrochloric acid or phosphoric acid, or with an organic acid e.g., acetic acid or maleic acid.

The amino acid residues described herein are in the “L” isomeric form, unless otherwise indicated. However, residues in the “D” isomeric form can be substituted for any L-amino acid residue, as long as the peptide substantially retains the desired antibody specificity.

Suitable analogs may be readily synthesized by now-standard peptide synthesis methods and apparatus or recombinant methods. All such analogs will essentially be based on the antigens of the invention as regards their amino acid sequence but will have one or more amino acid residues deleted, substituted or added. When amino acid residues are substituted, such conservative replacements which are envisaged are those which do not significantly alter the structure or antigenicity of the polypeptide. For example basic amino acids will be replaced with other basic amino acids, acidic ones with acidic ones and neutral ones with neutral ones. In addition to analogs comprising conservative substitutions as detailed above, analogs comprising non-conservative amino acid substitutions are further contemplated, as long as these analogs are immunologically cross reactive with an antigen of the invention.

In other aspects, there are provided nucleic acids encoding these peptides, vectors comprising these nucleic acids and host cells containing them. These nucleic acids, vectors and host cells are readily produced by recombinant methods known in the art (see, e.g., Sambrook et al., 2001). For example, an isolated nucleic acid sequence encoding an antigen of the invention can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional peptide of the present invention.

According to the principles of the invention the kits comprise a plurality of antigens also referred to herein as antigen probe sets. These antigen probe sets comprising a plurality of antigens are reactive specifically with the sera of subjects having SLE. In some embodiments, the antigen probe sets are reactive specifically also with the sera of subjects having scleroderma. According to the principles of the invention, the plurality of antigens may advantageously be used in the form of an antigen array. According to some embodiments the antigen array is conveniently arranged in the form of an antigen chip. In other embodiments, the kit may further comprise means for determining the reactivity of antibodies in a sample to the plurality of antigens. For example, the kit may contain reagents, detectable labels and/or containers which may be used for measuring specific binding of antibodies to the antigen probes of the invention. In a particular embodiment, said kit is in the form of an antigen array. In some embodiments, said kit comprises means for comparing reactivity patterns of antibodies in different samples to the plurality of antigens. In other embodiments, said kit may further comprise negative and/or positive control samples.

For example, a negative control sample may contain a sample from at least one healthy individual (e.g., an individual not-afflicted with SLE). A positive control may contain a sample from at least one individual afflicted with SLE, or a subtype of SLE which is being diagnosed. Other non-limiting examples are a panel of control samples from a set of healthy individuals or diseased individuals, or a stored set of data from control individuals.

Antibodies, Samples and Immunoassays

Antibodies, or immunoglobulins, comprise two heavy chains linked together by disulfide bonds and two light chains, each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CH1). The variable domains of each pair of light and heavy chains form the antigen binding site.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)2 fragments. Further included within the scope of the invention (for example as immunoassay reagents, as detailed herein) are chimeric antibodies; recombinant and engineered antibodies, and fragments thereof.

Exemplary functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens. An “antigenic peptide” is a peptide which is capable of specifically binding an antibody.

In another embodiment, detection of the capacity of an antibody to specifically bind an antigen probe may be performed by quantifying specific antigen-antibody complex formation. The term “specifically bind” as used herein means that the binding of an antibody to an antigen probe is not competitively inhibited by the presence of non-related molecules.

In certain embodiments, the method of the present invention is performed by determining the capacity of an antigen of the invention to specifically bind antibodies of the IgG isotype, or, in other embodiments, antibodies of the IgM, isolated from a subject.

Methods for obtaining suitable antibody-containing biological samples from a subject are well within the ability of those of skill in the art. Typically, suitable samples comprise whole blood and products derived therefrom, such as plasma and serum. In other embodiments, other antibody-containing samples may be used, e.g. CSF, urine and saliva samples.

Numerous well known fluid collection methods can be utilized to collect the biological sample from the subject in order to perform the methods of the invention.

In accordance with the present invention, any suitable immunoassay can be used with the subject peptides. Such techniques are well known to the ordinarily skilled artisan and have been described in many standard immunology manuals and texts. In certain preferable embodiments, determining the capacity of the antibodies to specifically bind the antigen probes is performed using an antigen probe array-based method. Preferably, the array is incubated with suitably diluted serum of the subject so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe.

The Antigen Chip

Antigen microarrays are recently developed tools for the high-throughput characterization of the immune response (Robinson et al., 2002, Nat Med 8, 295-301), and have been used to analyze immune responses in vaccination and in autoimmune disorders (Robinson et al., 2002; Robinson et al., 2003, Nat Biotechnol. 21, 1033-9; Quintana et al., 2004; Kanter et al., 2006, Nat Med 12, 138-43). It has been hypothesized, that patterns of multiple reactivities may be more revealing than single antigen-antibody relationships (Quintana et al., 2006, Lupus 15, 428-30) as shown in previous analyses of autoimmune repertoires of mice (Quintana et al., 2004; Quintana et al., 2001, J Autoimmun 17, 191-7) and humans (Merbl et al., 2007, J Clin Invest 117, 712-8; Quintana et al., 2003, J Autoimmun 21, 65-75) in health and disease. Thus, autoantibody repertoires have the potential to provide both new insights into the pathogenesis of the disease and to serve as immune biomarkers (Cohen, 2007, Nat Rev Immunol. 7, 569-74) of the disease process.

According to some aspects the methods of the present invention may be practiced using antigen arrays as disclosed in WO 02/08755 and U.S. 2005/0260770 to some of the inventors of the present invention, the contents of which are incorporated herein by reference. WO 02/08755 is directed to a system and an article of manufacture for clustering and thereby identifying predefined antigens reactive with undetermined immunoglobulins of sera derived from patient subjects in need of diagnosis of disease or monitoring of treatment. Further disclosed are diagnostic methods, and systems useful in these methods, employing the step of clustering a subset of antigens of a plurality of antigens, said subset of antigens being reactive with a plurality of antibodies being derived from a plurality of patients, and associating or disassociating the antibodies of a subject with the resulting cluster.

U.S. Pat. App. Pub. No. 2005/0260770 to some of the inventors of the present invention discloses an antigen array system and diagnostic uses thereof. The application provides a method of diagnosing an immune disease, particularly diabetes type 1, or a predisposition thereto in a subject, comprising determining a capacity of immunoglobulins of the subject to specifically bind each antigen probe of an antigen probe set. The teachings of said disclosures are incorporated in their entirety as if fully set forth herein.

In other embodiments, various other immunoassays may be used, including, without limitation, enzyme-linked immunosorbent assay (ELISA), flow cytometry with multiplex beads (such as the system made by Luminex), surface plasmon resonance (SPR), elipsometry, and various other immunoassays which employ, for example, laser scanning, light detecting, photon detecting via a photo-multiplier, photographing with a digital camera based system or video system, radiation counting, fluorescence detecting, electronic, magnetic detecting and any other system that allows quantitative measurement of antigen-antibody binding.

Various methods have been developed for preparing arrays suitable for the methods of the present invention. State-of-the-art methods involves using a robotic apparatus to apply or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Conveniently, the glass surface is first activated by a chemical treatment that leaves a layer of reactive groups such as epoxy groups on the surface, which bind covalently any molecule containing free amine or thiol groups. Suitable supports may also include silicon, nitrocellulose, paper, cellulosic supports and the like.

Preferably, each antigen probe, or distinct subset of antigen probes of the present invention, which is attached to a specific addressable location of the array is attached independently to at least two, more preferably to at least three separate specific addressable locations of the array in order to enable generation of statistically robust data.

In addition to antigen probes of the invention, the array may advantageously include control antigen probes or other standard chemicals. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided by the signal (e.g., fluorescence intensity) from the normalization control probes thereby normalizing the measurements. Normalization control probes can be bound to various addressable locations on the antigen probe array to control for spatial variation in antibody-ligand probe efficiency. Preferably, normalization control probes are located at the corners or edges of the array to control for edge effects, as well as in the middle of the array.

The labeled antibody ligands may be of any of various suitable types of antibody ligand. Preferably, the antibody ligand is an antibody which is capable of specifically binding the Fc portion of the antibodies of the subject used. For example, where the antibodies of the subject are of the IgM isotype, the antibody ligand is preferably an antibody capable of specifically binding to the Fc region of IgM antibodies of the subject.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. Suitable fluorophore-conjugated antibodies specific for antibodies of a specific isotype are widely available from commercial suppliers and methods of their production are well established.

Antibodies of the subject may be isolated for analysis of their antigen probe binding capacity in any of various ways, depending on the application and purpose. While the subject's antibodies may be suitably and conveniently in the form of blood serum or plasma or a dilution thereof (e.g. 1:10 dilution), the antibodies may be subjected to any desired degree of purification prior to being tested for their capacity to specifically bind antigen probes. The method of the present invention may be practiced using whole antibodies of the subject, or antibody fragments of the subject which comprises an antibody variable region.

Data Analysis

Advantageously, the methods of the invention may employ the use of learning and pattern recognition analyzers, clustering algorithms and the like, in order to discriminate between reactivity patterns of healthy control subjects to those of patients having SLE or scleroderma. As such, this term specifically includes a difference measured by, for example, determining the reactivity of antibodies in a test sample to a plurality of antigens, and comparing the resulting reactivity pattern to the reactivity patterns of negative and positive control samples (e.g. samples obtained from control subjects which are not afflicted with SLE or patients afflicted with SLE, respectively) using such algorithms and/or analyzers. The difference may also be measured by comparing the reactivity pattern of the test sample to a predetermined classification rule obtained in such manner.

In some embodiments, the methods of the invention may employ the use of learning and pattern recognition analyzers, clustering algorithms and the like, in order to discriminate between reactivity patterns of subjects having a subtype of SLE to control subjects. For example, the methods may include determining the reactivity of antibodies in a test sample to a plurality of antigens, and comparing the resulting pattern to the reactivity patterns of negative and positive control samples using such algorithms and/or analyzers.

Thus, in another embodiment, a significant difference between the reactivity pattern of a test sample compared to a reactivity pattern of a control sample, wherein the difference is computed using a learning and pattern recognition algorithm, indicates that the subject is afflicted with SLE. For example, the algorithm may include, without limitation, supervised or non-supervised classifiers including statistical algorithms including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor, artificial neural networks, coupled two-way clustering algorithms, multi-layer perceptrons (MLP), generalized regression neural network (GRNN), fuzzy inference systems (FIS), self-organizing map (SOM), genetic algorithms (GAS), neuro-fuzzy systems (NFS), adaptive resonance theory (ART).

In certain embodiments, one or more algorithms or computer programs may be used for comparing the amount of each antibody quantified in the test sample against a predetermined cutoff (or against a number of predetermined cutoffs). Alternatively, one or more instructions for manually performing the necessary steps by a human can be provided.

Algorithms for determining and comparing pattern analysis include, but are not limited to, principal component analysis, Fischer linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes Similar to statistical analysis revealing underlying patterns in a collection of data, neural networks locate consistent patterns in a collection of data, based on predetermined criteria.

Suitable pattern recognition algorithms include, but are not limited to, principal component analysis (PCA), Fisher linear discriminant analysis (FLDA), soft independent modeling of class analogy (SIMCA), K-nearest neighbors (KNN), neural networks, genetic algorithms, fuzzy logic, and other pattern recognition algorithms. In some embodiments, the Fisher linear discriminant analysis (FLDA) and canonical discriminant analysis (CDA) as well as combinations thereof are used to compare the output signature and the available data from the database.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

Principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

In another embodiment, the algorithm is a classifier. One type of classifier is created by “training” the algorithm with data from the training set and whose performance is evaluated with the test set data. Examples of classifiers used in conjunction with the invention are discriminant analysis, decision tree analysis, receiver operator curves or split and score analysis.

The term “decision tree” refers to a classifier with a flow-chart-like tree structure employed for classification. Decision trees consist of repeated splits of a data set into subsets. Each split consists of a simple rule applied to one variable, e.g., “if value of “variable 1” larger than “threshold 1”; then go left, else go right”. Accordingly, the given feature space is partitioned into a set of rectangles with each rectangle assigned to one class.

The terms “test set” or “unknown” or “validation set” refer to a subset of the entire available data set consisting of those entries not included in the training set. Test data is applied to evaluate classifier performance.

The terms “training set” or “known set” or “reference set” refer to a subset of the respective entire available data set. This subset is typically randomly selected, and is solely used for the purpose of classifier construction.

Diagnostic Methods

As used herein the term “diagnosing” or “diagnosis” refers to the process of identifying a medical condition or disease (e.g., SLE) by its signs, symptoms, and in particular from the results of various diagnostic procedures, including e.g. detecting the reactivity of antibodies in a biological sample (e.g. serum) obtained from an individual, to a plurality of antigens. Furthermore, as used herein the term “diagnosing” or “diagnosis” encompasses screening for a disease, detecting a presence or a severity of a disease, distinguishing a disease from other diseases including those diseases that may feature one or more similar or identical symptoms, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations.

In one embodiment, the subject being diagnosed according to the methods of the invention is symptomatic. In other embodiments, the subject is asymptomatic.

The diagnostic procedure can be performed in vivo or in vitro, preferably in vitro.

According to some embodiments, the invention provides diagnostic methods useful for the detection of SLE or scleroderma.

In some embodiments, the methods of the invention are useful in diagnosing systemic lupus erythematosus (SLE) or lupus. “Lupus” as used herein is an autoimmune disease or disorder involving antibodies that attack connective tissue.

In an additional embodiment, the present invention provides a method of treating a subject having SLE, comprising determining SLE in the subject by the methods of the invention, and administering to said subject a therapeutic effective amount of a medicament for SLE, thereby treating SLE.

Criteria for Diagnosing SLE

The 1982 American College of Rheumatology (ACR) criteria summarize features necessary to diagnose SLE. The presence of 4 of the 11 criteria yields a sensitivity of 85% and a specificity of 95% for SLE. Patients with SLE may present with any combination of clinical features and serologic evidence of lupus.
Serositis—Pleurisy, pericarditis on examination or diagnostic ECG or imaging
Oral ulcers—Oral or nasopharyngeal, usually painless; palate is most specific
Arthritis—Nonerosive, two or more peripheral joints with tenderness or swelling
Photosensitivity—Unusual skin reaction to light exposure
Blood disorders—Leukopenia (<4×10³ cells/μL on more than one occasion), lymphopenia (<1500 cells/μL on more than one occasion), thrombocytopenia (<100×10³ cells/μL in the absence of offending medications), hemolytic anemia
Renal involvement—Proteinuria (>0.5 g/d or 3+positive on dipstick testing) or cellular casts
ANAs—Higher titers generally more specific (>1:160); must be in the absence of medications associated with drug-induced lupus
Immunologic phenomena—dsDNA; anti-Smith (Sm) antibodies; anti-phospholipid antibodies (anticardiolipin immunoglobulin G [IgG] or immunoglobulin M [IgM] or lupus anticoagulant); biologic false-positive serologic test results for syphilis, lupus erythematosus (LE) cells (omitted in 1997)
Neurologic disorder—Seizures or psychosis in the absence of other causes
Malar rash—Fixed erythema over the cheeks and nasal bridge, flat or raised
Discoid rash—Erythematous raised-rimmed lesions with keratotic scaling and follicular plugging, often scarring

Two of the most commonly used instruments for SLE diagnosis are the Systemic Lupus Erythematosus Disease Activity Index (SLEDAI) and the Systemic Lupus Activity Measure (SLAM).

SLE Disease Activity Index (SLEDAI)

The SLEDAI is an index that measures disease activity by weighting the importance of each organ system involved. The SLEDAI includes 24 items, representing nine organ systems. The variables are obtained by history, physical examination and laboratory assessment. Each item is weighted from 1 to 8 based on the significance of the organ involved. For example, mouth ulcers are scored as 2, while seizures are scored as 8. The laboratory parameters that are included in the SLEDAI include white blood cell count, platelet count, urinalysis, serum C3, C4 and anti-dsDNA. The total maximum score is 105.

Systemic Lupus Activity Measure (SLAM)

The SLAM includes 32 items representing 11 organ systems. The items are scored not only as present/absent, but graded on a scale of 1 to 3 based on severity. The total possible score for the SLAM is 86. Both the SLEDAI and the SLAM have been shown to be valid, reliable, and sensitive to change over time (Liang et al. 1989, Arth Rheum 32:1107-18), and are widely used in research protocols and clinical trials. These indices are particularly useful for examining the value of newly proposed serologic or inflammatory markers of disease activity in SLE.

Despite the obvious utility of these instruments, there are some drawbacks. First, there is not always complete agreement between the SLAM and the SLEDAI in the same set of patients. There are several possible reasons for these discrepancies. Unlike the SLEDAI, the SLAM includes constitutional symptoms such as fatigue and fever, which may or may not be considered attributable to active SLE; this activity index relies on physician interpretation. In addition, the SLEDAI does not capture mild degrees of activity in some organ systems and does not have descriptors for several types of activity, such as hemolytic anemia.

The following examples are presented in order to more fully illustrate some embodiments of the invention. They should, in no way be construed, however, as limiting the broad scope of the invention.

### EXAMPLES

### Materials and Methods

Human Subjects

The study was approved by the Institutional Review Boards of each participating clinical unit; informed consent was obtained from all participants. Sera from 49 SLE patients (all fulfilled the American College of Rheumatology criteria for SLE), 24 scleroderma patients, 8 pemphigus patients and 23 healthy controls were studied. Blood samples and clinical data were collected from patients arriving at the rheumatology and nephrology unit at Rabin Medical Center, Petach Tikva, Israel; and the rheumatology unit and the hematology department of the Sheba Medical Center, Israel and the department of dermatology, Tel Aviv Sourasky Medical Center.

Antigen Microarrays and Serum Testing

Antigen microarray chips were prepared as previously described (Quintana et al. Lupus. 2006; 15:428-30). Briefly, 64 antigens, some in several concentrations or in different solvents (overall 110 different preparations), were spotted in triplicates on epoxy-activated glass substrates using a 48-pin robot (Microgrid 600; Genomics Solutions, Ann Arbor, Mich.). These antigens included proteins, synthetic peptides from the sequences of selected proteins, nucleotides, phospholipids, and other self and non-self molecules (as listed below). The microarrays were then blocked for 1 hr at 37° with 1% bovine serum albumin Test serum in 1% bovine serum albumin blocking buffer (1:10 dilution) was incubated under a coverslip for 1 hr at 37°. The arrays were then washed and incubated for 1 hr at 37° with a 1:500 dilution of two detection antibodies, mixed together: a goat anti-human IgG Cy3-conjugated antibody, and a goat anti-human IgM Cy5-conjugated antibody (Jackson ImmunoResearch Laboratories Inc., West Grove, Pa.). Image acquisition was performed by laser (Agilent Technologies, Santa Clara, Calif.) and the results were analyzed using Quantarray software (Packard BioChip Technologies, Billerica, Mass.) and software developed by the current inventors. The quantitative range of signal intensity of binding to each antigen spot was 0-65,000; this range of detection made it possible to obtain reliable data at a 1:10 dilution of test samples.

Following is a list of the antigens used in the example section: Actin (actin from bovine muscle, A3563, Sigma; beta2GP1-A2299-77E, US Biological; BMP4 (Bone morphogenic protein 4)—Human recombinant, CYT-36, Prospec; Buserelin-HOR-255, Prospec; Cardiolipin-C0563, Sigma; CD99-Human recombinant, PRO-294, Prospec; Centromere A-Human recombinant, PRO-389 Prospec; Centromere B-Human recombinant, PRO-390 Prospec; CMV (Cytomegalovirus) Pp150-recombinant, CMV-216, Prospec; Collagen III C4407, Sigma; Collagen IV C7521, Sigma; DNA (cytosine-5) methyltransferase 1-recombinant, ab91367, abcam; dsDNA-D1501, Sigma; ssDNA-D8899, Sigma; DSG (Desmoglein) 1-Human recombinant, H00001828-P01, Abnova; DSG (Desmoglein)3-ab87441, abcam; EBNA1(EBV nuclear antigen 1)-recombinant, EBV-276, Prospec; EBV (Epstein-Barr virus)p18-recombinant, EBV-273, Prospec; EBV p23-recombinant, EBV-278, Prospec; EBVEA (Epstein-Barr virus early antigen)-recombinant, EBV-272, Prospec; FABP 3 (Fatty Acid Binding Protein 3)-recombinant, PRO-340, Prospec; Fibrinogen-F4753, Sigma; FOX (Forkhead box) Protein 3-p290-304 (TKASSVASSQGPVVP, UniProtKB:B7ZLG1, this sequence has a 60% overlap with the matching human TKASSVASSDKGSCC; GLP1 (Glucagon Like Peptide-1)-recombinant, HOR-236, Prospec; GROa (Growth regulated protein alpha)-recombinant, CHM-329, Prospec; GST (Glutathione-S-Transferase)-G8642, Sigma; HGF (Hepatocyte growth factor)-recombinant, CYT-244, Prospec; Horseradish Peroxidase-P6782, Sigma; HSP60 amino acids 21-41, QSIVPALEIANAHRKPLVIIA, UniProtKB:Q53QD5; hsp60 amino acids 240-259, QDAYVLLSEKKOSSVQSIVP, UniProtKB:P10809, this sequence has a 90% overlap with the matching human QDAYVLLSEKKISSIQSIVP; HSP60p26-amino acids 376-395, EQLDITTSEYEKEKLNERLA, UniProtKB: P63038; HSP60-amino acids 436-455, IVLGGGCALLRCIPALDSLK, UniProtKB:P63038; Hyaluronic acid from rooster comb-H5388, Sigma; Hyaluronic acid sodium salt, from Streptococcus Equi-53747, Sigma; Hyaluronic Acid human-H1504, Sigma; IGFBP1 (Insulin growth factor binding protein 1)-recombinant, CRI232B, Cellsciences; IgG-12511, Sigma; IgM-I8260, Sigma; La-recombinant, PRO-327, Prospec; Lipopolysaccharides from Pseudomonas Aeruginosa-L9143, Sigma; Lipopolysaccharides from Salmonella Enterica-L5886, Sigma; Lysosomal membrane protein 2-recombinent, H00003 920, Abnova; Methyl-CpG-binding domain protein 2-recombinant, ab40707, Abcam; Methyl-CpG-binding domain protein 4-recombinant, H00008930, Abnova; MOG (Myelin Oligodendrocyte Glycoprotein)-p35-55-PRO-371, Prospec; MPO (Myeloperoxidase)-ENZ-074, Prospec; NRMJ amino acids 206-234 LGCSSRGVCVDGQCICDSE, UniProtKB: F1LQ63; P278 (HSP60 amino acids 458-474)-NEDQKIGIEIIKRALKI UniProtKB: P63038; p53 p10-amino acids 14-33, KTCPVQLWVSATPPAGSRVR; UniProtKB: A5JTV6; p53p11-amino acids 29-48-GSRVRAMAIYKKSQHMTEVV, UniProtKB:A5JTV6; p53 amino acids 253-272-DSSGNLLGRDSFEVRVCACP; UniProtKB: P02340; p53aminoacids 53-72, LPQDVEEFFEGPSEALRVSG, UniProtKB: P02340; PCNA (Proliferating cell nuclear antigen)-recombinant, PRO-303, Prospec; PDGF Receptor (platelet-derived growth factor receptor)-recombinant, D0946, Sigma; PDI (Protein Disulfide Isomerase)-recombinant, ENZ-262, Prospec; Pneumoccocal capsular polysaccharide type 4-purchased from ATCC (Manassas, Va.); PR3 (Proteinase 3)-CSI14825A, Cellsciences; R060-recombinant, PRO-329, Prospec; SAP90 (Disks large homolog 4) amino acids 63-82-VDVREVTHSAAVEALKEAGS UniProtKB:K7EKU8; Sm (Smith antigen)-CSI14863, Cellsciences; SYPH (synaptophysin; rat) amino acids 81-100-CVKGGTTKIFLVGDYSSSAE, UniProtKB: P07825; Thyroglobulin-T1001, Sigma; Topoisomerase 1-recombinant, ENZ-306, Prospec; and U1RNP (U1 ribonucleoprotein complex)-recombinant, PRO-445, Prospec.

Image Analysis and Data Processing

The foreground and background intensities of multiple spots of each antigen were averaged, and a log-base-10 value of the difference between the foreground and the background was calculated; differences <500 were clamped to 500 and then log transformed. To control for differences between different slides, the average laser intensity value of each slide (in the corresponding IgM or IgG channel) was then subtracted. The value of each antigen was then shifted such that its minimal value over the entire data set equaled zero. The resulting value was taken as the antigen reactivity of the antibodies binding to that spotted antigen. Antigens that showed zero reactivity in more than 80% of the slides were excluded, as were antigens whose coefficient of variation across slides was lower than 20%.

Statistical Analysis

The inventors sought to identify antigens whose reactivity is higher or lower in a specific study subgroup compared to other subgroups. An antigen i was determined to characterize study subgroup A with respect to subgroup B, if at least 20% of the subjects in subgroup A manifested reactivity higher than a given threshold, which was set at a Positive Predictive Value (PPV) of 90%—in other words, the rank order of reactivities to the particular antigen showed that 90% or more of the highest reactivities belonged to subgroup A relative to subgroup B subjects. Subjects who manifested reactivity higher than that threshold were termed ‘positive’ and antigen i was considered to be ‘increased’ in subgroup A. The same procedure was performed in the case that group A manifested lower reactivity than group B, namely at least 20% of the subjects in subgroup A showed reactivity lower than the threshold level set at a PPV of 90%—in other words, at least 90% of the lowest reactivities belonged to subgroup A compared to subgroup B. Subjects for which reactivity was lower than threshold were termed ‘positive’ and antigen i was declared as ‘decreased’ in subgroup A. The cutoff and positivity were determined specifically for each antigen and for a specific analysis, for example, SLE vs. SSc, or SLE vs. healthy controls.

P-values were calculated via randomization and were subjected to multiple comparisons correction. All ‘decreased’ cases passed a false discovery rate (FDR) of up to 10%.

Antigens that were ranked as ‘increased’ with a sensitivity score of at least 30% passed the FDR test. However, due to the over-representation of SLE specimens compared to SSc and to the healthy controls, some of the ‘increased’ antigens that manifested a sensitivity score below 30% did not pass the 10% FDR level. Nevertheless, these antigens were included in the data since ‘positive’ slides for such antigens overlapped with slides which were ‘positive’ for dsDNA (corresponding p-values were smaller than 8·10⁻³, for a FDR level of 5%).

### Example 1

### IgG and IgM Reactivities in SLE Patients Compared to Those of Healthy Controls and Scleroderma Patients

Table 1 shows antigen reactivities with PPV≧90% of the IgG and IgM isotypes that were either elevated or decreased in the sera of the SLE patients compared to the reactivities of scleroderma patients and healthy controls. IgG reactivities were found to be increased for known SLE antigens such as DNA, Sm, β2GP1 and La, in addition to other antigens. Increased IgG reactivities to HA from both human and streptococcus were prominent in SLE patients. Reactivities to EBV EA and EBVp23 were found to be increased in SLE patients, compared to healthy controls, but not compared to scleroderma patients.

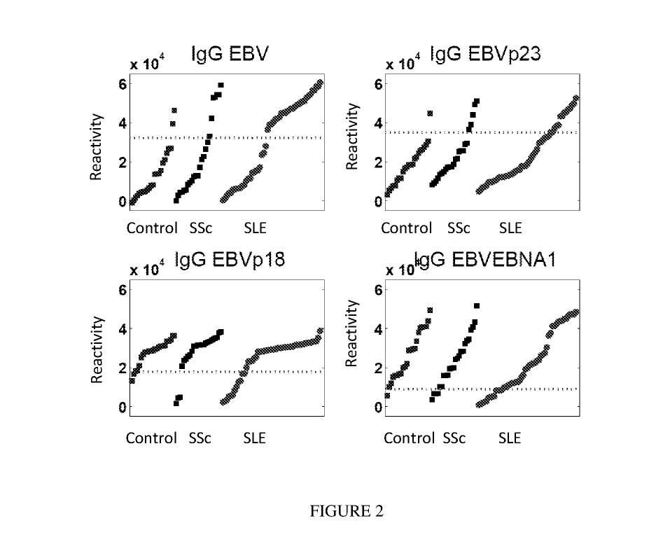

IgG reactivities to EBVp18 and EBNA-1 were found to be present in most healthy subjects; but unexpectedly, several of the SLE and scleroderma patients were both found to have decreased reactivities to these EBV antigens (Table 1 and FIG. 2).

IgM reactivities that characterized SLE patients compared to controls usually did not differ significantly when compared to scleroderma patients. Nevertheless, SLE patients showed increased IgM reactivities for B2GP1 compared to scleroderma patients. The IgG reactivities that distinguished between SLE and healthy controls also tended to discriminate between the SLE and scleroderma patients (Table 1). Increases in IgM reactivities were most prominent to DNA and HA, but also to FOXp3-p22, buserelin, MOG, BMP4 and Sm. IgM and IgG reactivities to dsDNA overlapped; 18 of 23 (78%) SLE patients positive for IgM anti-dsDNA were also positive for IgG anti-dsDNA. In addition, the significant IgM and IgG reactivities were found to overlap: 5 of the 6 antigens significant for IgM reactivity were also significant for IgG reactivity (Table 1).

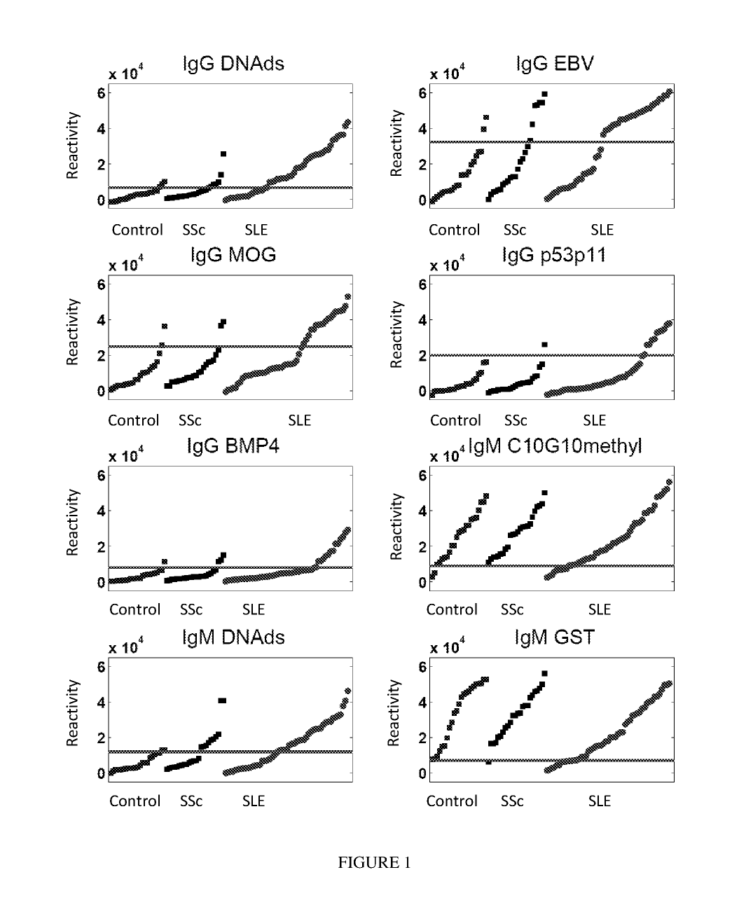

IgM reactivities to GST were found to be high in all the study groups, but a subgroup of SLE patients were found to have decreased reactivities compared to controls and Scleroderma patients (FIG. 1).

IgM reactivities to EBVEA were found to be decreased in SLE patients. Although the requirement of PPV≧90% was not met, SLE mean reactivities were decreased by 44% compared to controls and by 37% compared to scleroderma patients (P<0.05) (FIG. 1).

In general, the different subgroups of SLE patients with increases or decreases in IgM and IgG reactivities partially overlapped each other; no reactivities or lack of reactivities were correlated in any subgroup. No clear correlation was found between the increases or decreases in IgM or IgG reactivities and the clinical manifestations of the disease. The SLE antibody profile overlapped that of the scleroderma patients with regard to EBV antigens but was significantly different with regard to other antigens; both groups of patients differed significantly from healthy controls in their antibodies both to EBV antigens and other antigens.

TABLE 1
Sensitivity of antibody reactivities in SLE patients compared to
healthy controls and scleroderma patients.

| | Sensitivity (%) for PPV ≧90% | |
|---|---|---|
| | SLE compared | SLE compared to |
| Antigen | to controls | Scleroderma |

Increase in IgM

| | | |
|---|---|---|
| dsDNA | 47 | NS |
| HA (human) ** | 48 | NS |
| ssDNA | 40 | NS |
| hsp60p18 | 37 | 20 |
| Buserelin ** | 27 | NS |
| FOXp3-p22 ** | 39 | NS |
| Sm** | 29 | NS |
| MOG (myelin oligo- | 24 | NS |
| dendrocyte) ** | | |
| BMP4 (Bone morphogenic | 20 | NS |
| protein) ** | | |
| β2GP1 | NS | 22 |

Decrease in IgM

| | | |
|---|---|---|
| GST | 22 | 43 |

Increase in IgG

| | | |
|---|---|---|
| ssDNA | 69 | 55 |
| dsDNA | 65 | 63 |
| EBVEA (early antigen) | 55 | NS |
| HA (streptococcus) *** | 55 | 43 |
| FOXp3-p22 *** | 41 | 39 |
| HA (human) *** | 40 | 33 |
| MOG *** | 35 | 24 |
| BMP4 *** | 34 | 30 |
| hsp60-p26 *** | 29 | 29 |
| EBVp23 | 31 | NS |
| p53p11 *** | 20 | 20 |
| Sm | NS | 33 |
| p53p10 *** | 20 | 20 |
| IGFBP1 (Insulin growth factor | | |
| binding protein 1) *** | 20 | NS |
| β2GP1 | NS | 29 |
| HGF | NS | 30 |
| IgM | NS | 29 |
| La | NS | 22 |
| hsp60-p17a | NS | 20 |

Decrease in IgG

| | | |
|---|---|---|
| EBNA-1 | 22 | NS |
| EBVp18 | 20 | NS |

* NS = Non-significant.
** IgM significant antigens- Antigens other than DNA antigens that significantly characterize SLE patients.
*** IgG significant antigens- Antigens other than DNA or EBV antigens that significantly characterize SLE patients.

### Example 2

### A Subgroup of Anti-DNA Negative SLE Patients is Characterized by Reactivities to EBV Antigens

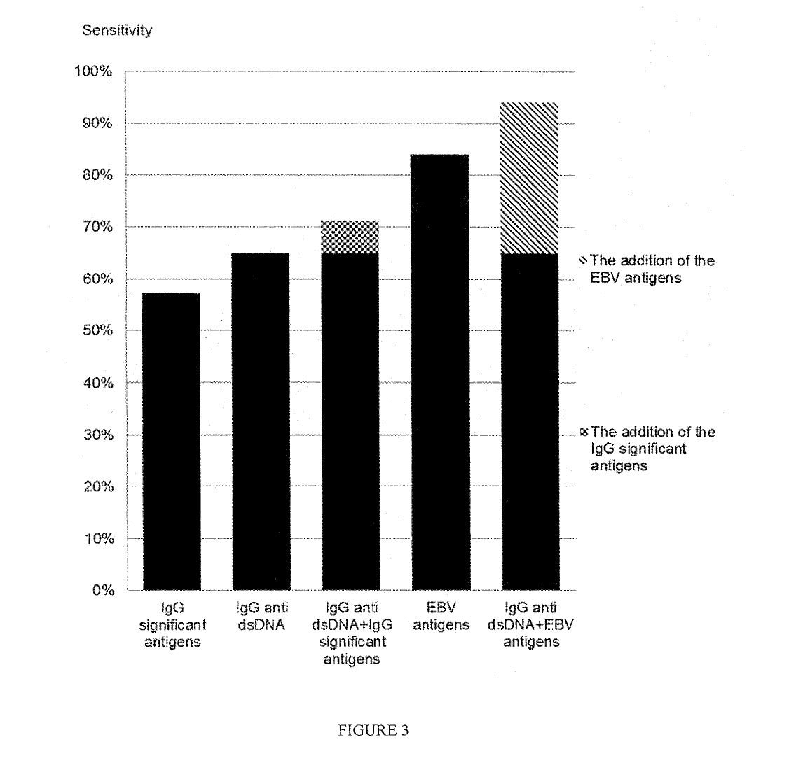

Autoantibodies to EBV antigens characterized 84% of SLE patients, and, unlike the reactivities to the antigens other than EBV, 29% of the SLE patients positive for EBV antigens were not detected by their anti-dsDNA reactivity. Hence, combining dsDNA and EBV antigens increased the serological detection of SLE to 94% (FIG. 3). Reactivity to EBV antigens thus contrast with the 59 other antigens, which failed to provide information that was not already provided by anti-dsDNA reactivity. No significant clinical difference was found between these different serological subgroups of SLE patients. Similarly, the reactivity to EBV antigens detected scleroderma (SSc) patients who were negative for dsDNA antibodies. Using the thresholds set by the SLE patients, 14 (58%) SSc patients were detected by the EBV antigens but only 2 of them were positive for dsDNA

### Example 3

### IgG and IgM Reactivities in Scleroderma Patients

Table 2 shows the percent sensitivities to antigens that were found to be increased in SSc patients compared to healthy controls and SLE patients. Note that only reactivities to Topoisomerase and Centromere B differed significantly in SSc patients compared to both healthy controls and SLE patients.

TABLE 2
Percent sensitivities in scleroderma patients compared to healthy
controls and SLE patients that passed PPV ≧90%
Increased reactivities in scleroderma patients compared to
healthy controls and SLE patients

Sensitivity (%) for PPV ≧90%

| | | |
|---|---|---|
| | Scleroderma compared | Scleroderma compared |
| Antigen | to healthy controls | to SLE |

Increased IgM

| | | |
|---|---|---|
| dsDNA | 33 | NS |
| Centromere B | 25 | NS |

Increased IgG

| | | |
|---|---|---|
| Topoisomerase | 50 | 33 |
| Centromere B | 46 | 25 |

Similar to the SLE patients, increases in IgG reactivities to EBVEA and EBVp23 and decreases in IgG reactivities to EBVp18 and EBNA1 were found in SSc patients compared to controls. The apparent lack of significance can be attributed to the requirement of PPV≧90% and the small number of SSc patients (FIG. 2).

### Example 4

### Increased IgG Reactivities to Other Antigens Characterize the SLE Patients Scoring Positive for IgG Anti-dsDNA

The inventors examined whether a combination of IgG reactivities to antigens other than EBV or dsDNA might increase the serologic detection of SLE patients. 9 IgG reactivities were identified that significantly characterized SLE patients compared to controls (i.e., HA (streptococcal), FOXp3-p22, HA (human), MOG, BMP4, HSP60-p26, p53-p10, p53p11 and IGFBP1; termed IgG significant antigens; Table 1). An SLE patient was classified as positive for IgG significant antigens if he or she were positive for at least 2 of the 9 antigens. Of the 49 SLE patients, 57% were detected by their IgG reactivities to IgG significant antigens; reactivity to dsDNA alone detected 65% of SLE patients. Indeed, the detection rate improved by only 6% by the addition of the IgG significant antigens to the dsDNA detection rate. Hence the information provided by the IgG significant antigens was mostly redundant to that provided by IgG anti-dsDNA (FIG. 3).

### Example 5

### Increased IgM Reactivities Characterize the SLE Patients Scoring Positive for IgM Anti-dsDNA

The inventors further investigated whether SLE patients manifest an overlap between IgM anti-dsDNA and IgM reactivities to the 6 antigens found to characterize SLE patients (i.e., HA (human), FOXp3-p22, Sm, buserelin, MOG and BMP4, termed IgM significant antigens; Table 1). An SLE patient was classified as positive for IgM significant antigens if he or she were IgM positive for at least 2 out of the 6 antigens. Of the 49 SLE patients, 43% were detected by their reactivities to IgM significant antigens; IgM reactivity to dsDNA alone detected 47% of SLE patients. Indeed, the detection rate improved by 8% by the addition of the IgM significant antigens to the IgM anti-dsDNA detection rate. Hence, similar to the overlap between IgG anti-dsDNA and the IgG significant antigens, the information provided by the reactivities to the IgM significant antigens was mostly redundant to that provided by IgM anti-dsDNA.

### Example 6

### SLE Patients can be Distinguished Serologically from SSc Patients

To distinguish SLE patients from SSc patients, IgG reactivities to dsDNA and IgM reactivities to GST were used to detect the SLE patients. Forty of the 49 SLE patients and 3 of the 24 SSc patients were detected in this way; however, 2 of the SSc patients and 1 SLE patient were positive for IgG to either Topoisomerase or Centromere B, and their diagnosis was changed to SSc; thus 1 SSc patient was left false positive for SLE and 39 SLE patients who were true positives. Overall, the combination of these 4 reactivities yielded a sensitivity and specificity of 80% and 96% respectively for detecting SLE patients (PPV=98%, NPV=70%).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of diagnosing an autoimmune disease selected from systemic lupus erythematosus (SLE) and scleroderma in a subject, the method comprising:
(i) determining the reactivity of IgG antibodies in a sample obtained from the subject to a plurality of antigens selected from EBVp18 and at least one antigen selected from EBVp23, EBNA-1 and EBVEA, thereby determining the reactivity pattern of the sample to the plurality of antigens; and
(ii) comparing the reactivity pattern of said sample to a control reactivity pattern;
wherein a significant difference between the reactivity pattern of said sample obtained from the subject compared to the reactivity pattern of a control sample is an indication that the subject is afflicted with SLE or scleroderma.

2. The method of claim 1, wherein the subject is negative for dsDNA antibodies.

3. The method of claim 1, wherein the plurality of antigens is used in the form of an antigen array.

4. The method of claim 1, wherein the plurality of antigens comprises EBVp23, EBVp18, and EBNA-1, and optionally EBVEA.

5. (canceled)

6. The method of claim 1, wherein said reactivity pattern of the sample comprises increased IgG reactivity, decreased IgG reactivity, or increased and decreased IgG reactivities.

7. (canceled)

8. (canceled)

9. The method of claim 6, wherein a reactivity pattern of the sample comprising increased IgG reactivity of at least one antigen selected from EBVp23 and EBVEA, compared to the reactivity pattern of the control sample, is an indication that the subject is afflicted with SLE or scleroderma.

10. The method of claim 6, wherein a reactivity pattern of the sample comprising decreased IgG reactivity is of at least one antigen selected from EBVp18 and EBNA-1 compared to the reactivity pattern of the control sample, is an indication that the subject is afflicted with SLE or scleroderma.

11. The method of claim 1, further comprising determining the reactivity of antibodies in said sample to at least one antigen selected from Glutathione S-Transferase (GST), FOXp3-p22, buserelin, MOG, HSP60-p26, P53-p10 and p53-p11, or a subset thereof.

12. The method of claim 11, wherein a reactivity pattern of the sample comprising significantly decreased IgM reactivity of GST; increased IgM reactivity of at least one antigen selected from FOXp3-p22, buserelin, MOG; or increased IgG reactivity of at least one antigen selected from FOXp3-p22, MOG, HSP60-p26, P53-p10 and p53-p11, compared to the reactivity pattern of a control sample, is an indication that the subject is afflicted with SLE.

13. A method of diagnosing SLE in a subject, the method comprising:
(i) determining the reactivity of IgG and IgM antibodies in a sample obtained from the subject to a plurality of antigens selected from the group consisting of EBVp18, EBVp23, GST, FOXp3-p22, buserelin and MOG, and a subset thereof; thereby determining the reactivity pattern of the sample to the plurality of antigens; and
(ii) comparing the reactivity pattern of said sample to a control reactivity pattern;
wherein a significant difference between the reactivity pattern of said sample obtained from the subject compared to the reactivity pattern of a control sample is an indication that the subject is afflicted with SLE.

14. The method of claim 13, wherein the plurality of antigens is selected from EBVp18 and at least one antigen selected from EBVp23, GST, FOXp3-p22, buserelin, MOG, HSP60-p26, P53-p10 and p53-p11.

15. The method of claim 13, wherein the plurality of antigens further comprises HSP60-p26, P53-p10 and p53-p11.

16. The method of claim 13, wherein a reactivity pattern of the sample comprising significantly increased IgG reactivity to at least one antigen selected from EBVp23, FOXp3-p22, MOG, HSP60-p26, P53-p10 and p53-p11, compared to the reactivity pattern of a control sample, is an indication that the subject is afflicted with SLE.

17. The method of claim 13, wherein a reactivity pattern of the sample comprising significantly decreased IgG reactivity to EBVp18, compared to the reactivity pattern of a control sample, is an indication that the subject is afflicted with SLE.

18. The method of claim 13, wherein a reactivity pattern of the sample comprising significantly increased IgM reactivity to at least one antigen selected from FOXp3-p22, buserelin and MOG, compared to the reactivity pattern of a control sample, is an indication that the subject is afflicted with SLE.

19. The method of claim 13, wherein a reactivity pattern of the sample comprising significantly decreased IgM reactivity to GST, compared to the reactivity pattern of a control sample, is an indication that the subject is afflicted with SLE.

20. (canceled)

21. (canceled)

22. The method of claim 13, wherein said plurality of antigens is used in the form of an antigen array.

23. A kit for the diagnosis of SLE or scleroderma in a subject comprising a plurality of antigens selected from the group consisting of: EBVp23, EBVp18, EBNA-1 and EBVEA or a subset thereof.

24. A kit for the diagnosis SLE in a subject comprising a plurality of antigens selected from the group consisting of: EBVp23, EBVp18, GST, FOXp3-p22, buserelin, MOG, or a subset thereof.

25. The kit of claim 24 wherein the plurality of antigens further comprises HSP60-p26, P53-p10 and p53-p11.

26. The kit of claim 23, wherein said kit is in the form of an antigen array.

27. The kit of claim 24, wherein said kit is in the form of an antigen array.

28.-32. (canceled)


SEQUENCE LISTING (18 sequences)

SEQ ID NO: 1 - protein, 71 aa, Epstein Barr Virus
ASAGTGALASSAPSTAVAQSATPSVSSSISSLRAATSGATAAASAAAAVDTGSGGGGQPHDTAPRGARKKQ

SEQ ID NO: 2 - protein, 161 aa, Epstein Barr Virus
SAPRKVRLPSVKAVDMSMEDMAARLARLESENKALKQQVLRGGACASSTSVPSAPVPPPEPLTARQREVMITQATGRLASQAMKKIEDKVRKSVDGVTTRNEMENILQNLTLRIQVSMLGAKGQPSPGEGTRPRESNDPNATRRARSRSRGREAKKVQISD

SEQ ID NO: 3 - protein, 90 aa, Epstein Barr Virus
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTG

SEQ ID NO: 4 - protein, 92 aa, Epstein Barr Virus
PVGEADYFEYHQEGGPDGEPDVPPGAIEQGPADDPGEGPSTGPRGQGDGGRRKKGGWFGKHRGQGGSNPKFENIAEGLRALLARSHVERTTD

SEQ ID NO: 5 - protein, 182 aa, Epstein Barr Virus
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGPVGEADYFEYHQEGGPDGEPDVPPGAIEQGPADDPGEGPSTGPRGQGDGGRRKKGGWFGKHRGQGGSNPKFENIAEGLRALLARSHVERTTD

SEQ ID NO: 6 - protein, 84 aa, Epstein Barr Virus
ASEPEDKSPRVQPLGTGLQQRPRHTVSPSPSPPPPPRTPTWESPARPETPSPAIPSHSSNTALERPLAVQLARKRTSSEARQKQ

SEQ ID NO: 7 - DNA, 20 bp, Epstein Barr Virus
cgcgcgcgcgcgcgcgcgcg

SEQ ID NO: 8 - protein, 218 aa, Homo sapiens
MPMILGYWDIRGLAHAIRLLLEYTDSSYEEKKYTMGDAPDYDRSQWLNEKFKLGLDFPNLPYLIDGAHKITQSNAILCYIARKHNLCGETEEEKIRVDILENQTMDNHMQLGMICYNPEFEKLKPKYLEELPEKLKLYSEFLGKRPWFAGNKITFVDFLVYDVLDLHRIFEPKCLDAFPNLKDFISRFEGLEKISAYMKSSRFLPRPVFSKMAVWGNK

SEQ ID NO: 9 - protein, 210 aa, Homo sapiens
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 10 - protein, 9 aa, Artificial Sequence
  Synthetic
EHWSYSLRP

SEQ ID NO: 11 - protein, 21 aa, Homo sapiens
MEVGWYRSPFSRVVHLYRNGK

SEQ ID NO: 12 - protein, 116 aa, Homo sapiens
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 13 - protein, 15 aa, Homo sapiens
TKASSVASSQGPVVP

SEQ ID NO: 14 - protein, 15 aa, Homo sapiens
TKASSVASSDKGSCC

SEQ ID NO: 15 - protein, 20 aa, Mus musculus
EQLDITTSEYEKEKLNERLA

SEQ ID NO: 16 - protein, 59 aa, Mus musculus
YSPPLNKLFCQLAKTCPVQLWVSATPPAGSRVRAMAIYKKSQHMTEVVRRCPHHERCSD

SEQ ID NO: 17 - protein, 20 aa, Mus musculus
KTCPVQLWVSATPPAGSRVR

SEQ ID NO: 18 - protein, 20 aa, Mus musculus
GSRVRAMAIYKKSQHMTEVV